(19) United States
(12) Patent Application Publication
(10) Pub. No.: US 2016/0053231 A1
(43) Pub. Date: Feb. 25, 2016

(54) TWO AND THREE DIMENSIONAL DECELLULARIZED ECM CONSTRUCTS AND USES THEREFOR

(71) Applicant: TUFTS UNIVERSITY, Medford, MA (US)

(72) Inventor: Qiaobing Xu, Somerville, MA (US)

(21) Appl. No.: 14/784,207

(22) PCT Filed: Apr. 9, 2014
(86) PCT No.: PCT/US14/33485

§ 371 Date: Oct. 13, 2015

Related U.S. Application Data
(60) Provisional application No. 61/810,404, filed on Apr. 10, 2013.

(51) Int. Cl. C12N 5/071; G01N 33/50

(52) U.S. Cl. CPC C12N 5/0697; G01N 33/5014; G01N 33/5088; C12N 2537/10; C12N 2533/90; C12N 2513/00

(57) ABSTRACT

A microfabricated multi-tissue system for in vitro drug toxicity testing having a plurality of layers, each of which is formed of decellularized tissue extracellular matrix (ECM) including parenchymal cells and non-parenchymal cells attached thereto. Also disclosed is a method for producing a decellularized ECM paste, and methods for producing an ECM construct and a porous 3-D scaffold from the decellularized ECM paste.

### BACKGROUND

1. Field of the Invention

This application relates to tissue-based engineered materials that can meet all mechanical, biological, and functional requirements for 3D cell scaffolds, in vitro drug toxicity testing, and for microfabricated multi-tissue culture systems.

2. Background Information

Current animal models and in vitro test systems are not highly predictive of the human response to toxic compounds. As a result, a majority of candidate drugs fail in human trials due to toxicity and lack of efficacy. The generation of in vitro models that can better mimic the circulatory system and human drug metabolism is necessary in order to provide a more accurate preclinical model and to reduce the number of animals used for drug testing.

3D cell-culture models provide a more physiologically relevant tissue structure which can recapitulate cell-cell and cell-matrix interactions while providing a more appropriate geometry for drug testing devices. Typically, several types of biodegradable and non-degradable polymers, such as poly(lactic-co-glycolic acid) and polystyrene, have been used to fabricate 3D cell scaffolds. However, synthetic polymer scaffolds lack endogenous factors that modulate cellular behavior.

The decellularization of extracellular matrix (ECM) is an attractive technique used to prepare scaffolds for tissue engineering applications. In the decellularization process, cells and cellular components are removed from native organs using ionic or nonionic detergents or enzymatic treatment. Decellularization eliminates the cells and cellular components of the organs while leaving surrounding structures such as the ECM and other structural molecules intact. This preserves the original tissue architecture, including the native microvasculature. Additionally, the mechanical properties of the ECM are also preserved. Decellularized organ matrices facilitate cell attachment, migration, and differentiation, ultimately leading to functional regeneration of the organs. Despite the promise of decellularized organ matrices for use in tissue engineering, it is difficult to regenerate from a whole decellularized organ a complex hierarchical organ structure in which many types of cells are organized into specialized tissues. This difficulty is caused, in part, by the requirement to seed cells into the decellularized organ matrix through perfusion techniques.

There is a need to develop new tissue-based engineered materials that can meet all mechanical, biological, and functional requirements for 3D cell scaffolds, in vitro drug toxicity testing, and for microfabricated multi-tissue culture systems.

### SUMMARY

To accomplish this goal, one aspect of this invention relates to a microfabricated multi-tissue system for in vitro drug toxicity testing. The system includes a plurality of layers, each of which is formed of decellularized tissue ECM and has a thickness of 10 μm to 2 mm At least one of the layers has parenchymal cells attached thereto and at least one other layer has non-parenchymal cells attached thereto. The layer including the parenchymal cells is stacked on the layer including the non-parenchymal cells thereby extending the survival of the parenchymal cells and maintaining their differentiated state.

Another aspect of this invention relates to an in vitro method for drug toxicity testing using the microfabricated multi-tissue system described above. The system is exposed to a test drug, and the viability of the parenchymal cells or the expression level of tissue-specific genes in the parenchymal cells after exposure to the test drug is determined The test drug is toxic if the viability of the parenchymal cells is reduced after exposure to the test drug or the expression level of tissue-specific genes is reduced as compared to a predetermined control expression level.

Still another aspect of this invention relates to a method for producing a three dimensional co-culture system. First, a tissue sample is sliced into a plurality of sections each having a thickness of 10 μm to 2 mm, followed by decellularizing the plurality of sections to form a plurality of sheets. At least one sheet is seeded with parenchymal cells and at least one sheet is seeded with non-parenchymal cells. The seeded sheets are cultured and then stacking to form a flat tube precursor. The precursor is then rolled to form a tubular structure having a lumen and an exterior surface.

Also within the scope of the invention is a microfabricated system for in vitro drug toxicity testing having a single layer formed of decellularized tissue ECM, the layer having a thickness of 10 μm to 2 mm The layer has parenchymal cells seeded therein.

A further aspect of this invention relates to a method for producing a reconstituted ECM construct. In this method, a decellularized ECM from a tissue is homogenized to form a paste. The paste is then cast into a mold to form a reconstituted ECM construct.

Also provided is a reconstituted ECM construct produced by the method described above.

In another aspect, a method for producing a porous reconstituted ECM scaffold is provided. The method includes the steps of homogenizing the decellularized ECM to form a paste, and lyophilizing the paste, thereby forming a porous reconstituted ECM scaffold.

A porous reconstituted ECM scaffold produced by the just-described method is also within the scope of the invention.

The details of one or more embodiments of the invention are set forth in the description and the drawings below. Other features, objects, and advantages of the invention will be apparent from the detailed description of several embodiments and also from the claims.

### DETAILED DESCRIPTION

In one embodiment, this invention relates to a microfabricated multi-tissue system for in vitro drug toxicity testing including a plurality of layers, each of which is formed of decellularized tissue ECM. In a particular embodiment, the plurality of layers is arranged in a tubular structure having a lumen and an exterior surface.

The tissues suitable for forming the layers include, but are not limited to, lung, liver, heart, kidney, intestine, tendon, pancreas, brain, skin, fat, cartilage, spleen, bone, and tumor. Each layer can be derived from a different tissue, or all layers can be derived from a single source tissue. The thickness of each layer can be 10 μm to 2 mm, e.g., 10 μm to 50 μm, 50 μm to 100 μm, 100 μm to 500 μM, 500 μM to 1 mm, and 1 mm to 2 mm.

The tissue mentioned above can be obtained from a mammal, e.g., human, pig, cow, dog, horse, rabbit, and rodent.

At least one layer of the multi-tissue system can be seeded with parenchymal cells, and at least one layer can be seeded with non-parenchymal cells. The parenchymal cells are organ-derived differentiated cells, e.g., hepatocytes, cardiomyocytes, kidney epithelial cells, enterocytes, beta cells, and cortical neurons. The non-parenchymal cells help to extend survival of and maintain the parenchymal cells in their differentiated state. The non-parenchymal cells can be macrophages, fibroblasts, epithelial cells, adipocytes, or endothelial cells. In a particular embodiment, the parenchymal cells are hepatocytes and the non-parenchymal cells are Kupffer cells, liver epithelial cells, hepatic stellate cells, or sinusoidal endothelial cells. In another preferred embodiment, the decellularized tissue ECM is derived from liver and the non-parenchymal cells are fibroblasts.

In certain embodiments, the source of decellularized tissue ECM and the source of parenchymal cells are the same. For example, if the decellularized tissue ECM is derived from liver, the parenchymal cells are hepatocytes. Alternatively, if the decellularized tissue ECM is derived from heart, the parenchymal cells are cardiomyocytes.

Decellularization can be accomplished using one or more decellularization agents selected from detergents, emulsification agents, proteases, and ionic strength solutions. For example, U.S. Pat. No. 6,962,814 describes suitable decellularization agents and conditions. Decellularization does not cause gross alteration in the structure of the tissue or substantial alteration in its biomechanical properties. The effects of decellularization on structure can be evaluated by light microscopy and/or ultrastructural examination. Preferably, following removal from the solution used in the decellularization, the decellularized tissue is washed in a physiologically appropriate solution, e.g., PBS or tissue culture medium. Washing removes residual decellularization solution that might otherwise cause deterioration of the decellularized tissue, inhibit the growth of subsequently seeded cells, and reduce biocompatibility.

In an embodiment, the microfabricated multi-tissue system described above can be used to carry out a method for in vitro drug toxicity testing. In the method, the system is exposed to a test drug, and the viability of the hepatocytes that were seeded in one or more layers of the system can be determined before and after exposure to the test drug. Viability can be measured, for example, by staining samples of the test system with 0.5 mg/ml 3-[4,5-dimethylthiazol-2-yl]-2,5 diphenyl tetrazolium bromide (MTT) and reading absorbance at 560 nm using a microtiter plate reader. Alternatively, the samples can be incubated with calcein AM and viable cells visualized in situ by confocal fluorescent microscopy. The test drug is considered to be toxic if the viability of the hepatocytes is reduced after exposure to the test drug. For example, a toxic drug can reduce the viability of the hepatocytes from 10-99%. Preferably, a toxic drug can reduce cell viability by 50%.

Alternatively, the expression level of certain hepatic genes in the hepatocytes can be determined before and after exposure to the test drug as a measure of the toxicity of the drug. For example, the enzymatic activity of UDP glucoronyl transferase and cytochrome P450 can be measured. Alternatively, the level of a hepatic specific protein or the expression level of the mRNA encoding it can be measured. For example, the amount of albumin, transferrin, pyruvate kinase, and urea can be determined before and after exposure to the test drug. The test drug is toxic if the expression level of the hepatic gene is reduced as compared to a predetermined control expression level. For example, the expression level of the hepatic gene can be reduced by 10% of the predetermined control level.

In the embodiment described supra in which the system is arranged in a tubular structure having a lumen and an exterior surface, the system can be exposed to the test drug via the lumen. Alternatively, the test drug can be added to a medium surrounding the system such that the drug can diffuse directly through the exterior surface into the outer ECM layer.

In an additional embodiment, more than one microfabricated multi-tissue system as described above can be combined so as to mimic the human body circulatory system. For example, a tissue system derived from different organs, such as heart, liver, lung, brain, and kidney, can be placed into separate containers which are connected via a tube. Upon adding a test compound to one container, any by-products produced by the tissue type in that container can then flow via the tube into a second container containing a different tissue type. In this way, toxicity of the by-products on the second tissue can be measured.

As mentioned above, within the scope of the invention is a method for producing a three dimensional co-culture system. First, a tissue sample is sliced into a plurality of sections. Each section can have a thickness of 10 μm to 2 mm, e.g., 10 μm to 2 mm, e.g., 10 μm to 50 μm, 50 μm to 100 μm, 100 μm to 500 μM, 500 μM to 1 mm, and 1 mm to 2 mm The preferred thickness is 100 μm to 500 μM. The tissue can be lung, liver, heart, kidney, intestine, tendon, pancreas, brain, skin, fat, cartilage, spleen, bone, and tumor. The tissue sections are decellularized to form a decellularized sheet. At least one decellularized sheet is then seeded with hepatocytes, and a second sheet is seeded with non-parenchymal cells. The non-parenchymal cells can be macrophages, fibroblasts, epithelial cells, adipocytes, and endothelial cells. In certain embodiments, the non-parenchymal cells can be Kupffer cells, liver epithelial cells, hepatic stellate cells, or sinusoidal endothelial cells. The seeded sheets are cultured, and the cultured sheets stacked together to form a flat tube precursor. The flat precursor is then rolled to form a tubular structure having a lumen and an exterior surface. Each sheet that is seeded with cells is seeded with a single cell type.

The decellularized ECM derived from various tissues as described above can also be used as a starting point to produce a reconstituted ECM construct. First, decellularized ECM is homogenized using a blender followed by a tissue homogenizer to break fibers in the decellularized ECM into smaller pieces, thereby forming an ECM paste. The tissue can be, but is not limited to, lung, liver, heart, kidney, intestine, tendon, pancreas, brain, skin, fat, cartilage, spleen, bone, or tumor.

The ECM paste can be cast into a mold to form the reconstituted ECM construct. The mold can be selected depending upon the desired shape and size of the construct. For example, the mold can be a 3D mold, a flat mold, or a micromold. The mold can be formed of paraffin or polydimethylsiloxane. In particular embodiments, the mold can be a 3D paraffin mold, a flat paraffin mold, and a polydimethylsiloxane micromold. In a specific embodiment, the micromold contains microchannels connecting discrete sections of the mold, forming a microfluidic system.

In another embodiment, the paste can be poured into a mold containing a linear wire array to form pores or microchannels. Each wire in the linear wire array can be 1 mm to 1 cm in diameter. After molding, the reconstituted ECM can be frozen and then lyophilized.

In yet another embodiment, the decellularized ECM, after homogenization, is further treated with a proteolytic enzyme that can cleave the ECM proteins. For example, trypsin, pepsin, proteinase K, elastase, and collagenase can be used to digest the homogenized ECM proteins. In a preferred embodiment, the homogenized ECM proteins are digested with pepsin.

Additionally, the reconstituted ECM paste can be mixed with a cross-linking agent prior to casting such that cross-linking occurs in the ECM paste during the molding process. The cross-linking agent can be, e.g., transglutaminase (TG), glutaraldehyde, genipin, and 1-(3-dimethylaminopropyl)-3-ethylcarbodiimide

Further, the decellularized ECM paste described above can be lyophilized to form a porous reconstituted ECM scaffold. Alternatively, a salt can be added to the ECM paste causing the formation of pores within the paste. The porous reconstituted ECM scaffold can be cross-linked by incubating it with a cross-linking agent, such as TG, glutaraldehyde, genipin, and 1-(3-dimethylaminopropyl)-3-ethylcarbodiimide. The porous reconstituted ECM scaffold can be seeded with cells in certain embodiments.

The porosity of the reconstituted ECM scaffold can be controlled by varying the concentration of the ECM in the paste. The size of the pores in the reconstituted ECM scaffold is directly correlated with the concentration of ECM in the ECM paste used to form the scaffold. The amount of ECM in the paste can vary from 0.1% (w/v) to 50% (w/v), preferably 0.5-2%.

Additionally, the reconstituted ECM scaffold described above can be mechanically flattened to form a sheet. For example, the reconstituted ECM scaffold can be compressed between a polytetrafluoroethylene (TEFLON( ) roller and a TEFLON block to form a flat sheet. The size of the flat sheet can be adjusted to any desired size. For example, the flat sheet can be cut to a size which can fit within a tissue culture dish.

Without further elaboration, it is believed that one skilled in the art can, based on the disclosure herein, utilize the present invention to its fullest extent. The following specific examples are, therefore, to be construed as merely descriptive, and not limitative of the remainder of the disclosure in any way whatsoever. All publications and patent documents cited herein are hereby incorporated by reference in their entirety.

### Example 1

### Preparation of a Reconstituted ECM Film

Bovine liver or heart tissue was cut into small pieces of <1 cm³ in volume using a razor blade, and then thoroughly rinsed in deionized (DI) water for 30 min to remove blood and cellular debris. The pieces were transferred into a decellularization solution composed of phosphate buffer saline (PBS), 1% sodium dodecyl sulfate (SDS), and 1% penicillin/streptomycin, and incubated with daily changes of the decellularization solution for 3-5 days to achieve complete decellularization. The decellularized tissue pieces were then incubated in DI water overnight to remove the SDS. The resulting decellularized ECM can be stored and preserved at −20° C.

Decellularized ECM was mechanically broken down into fine ECM fibers by blending followed by homogenizing using a tissue homogenizer to form a paste. The resulting ECM paste was either lyophilized and stored at −20° C. or further broken down into a gel-like material by incubating it for two days in a 2 mg/ml pepsin solution (2500 units/mg) containing 1 mM hydrochloric acid (HC1).

The ECM paste (0.5%-2% w/v) was mixed with 1% w/v TG and formed into a reconstituted ECM film by pressing it into a micropatterned polydimethylsiloxane (PDMS) micromold prepared using a standard soft lithography technique. After air-drying overnight, the reconstituted ECM film was peeled off from the PDMS micromold using a pair of tweezers.

### Example 2

### Preparation of a Porous Reconstituted ECM Scaffold

The ECM paste described above (0.5-2% w/v) was mixed with 1% w/v TG and frozen at −80° C. overnight. The frozen sample was lyophilized to remove the water component, thereby creating a porous 3D scaffold.

As an alternative to freeze-drying, salt leaching can be used to create a porous reconstituted ECM scaffold having a pore size dependent on the concentration of salt used.

### Example 3

### Porous Reconstituted ECM Scaffold having Macroscale Channels

ECM paste was prepared from bovine heart tissue as described above. After mixing the ECM paste with TG, the mixture was poured into a mold containing a linear wire array. The wires had a diameter of 250 μm. The molded ECM was frozen and then lyophilized After drying, the wire array template was removed from the scaffold, leaving behind pores or channels within the scaffold.

### Example 4

### Preparation of a 3D Co-Culture System

Frozen pieces of liver were sliced into sections 10 μm to 2 mm thick using a microtome and then decellularized as described above. Individual decellularized liver ECM sheets were seeded with fibroblasts or hepatocytes. The cell-seeded sheets were cultured for 1-2 days after which they were stacked together and rolled into tubular structures having a void in the center. This void acted as a lumen and facilitated nutrient and waste exchange. The tubular structure was cultured for 4-7 days to generate a stable 3D co-culture system useable for, e.g., drug toxicity testing.

### Example 5

### Cell Growth on a Reconstituted ECM Film

A reconstituted ECM film having micro-wells was produced as described above. The film was seeded with 5×10⁴ human hepatoma (HepG2) cells and the seeded film was centrifuged at 2000 rpm for 2 min such that the cells were deposited at the bottom of the micro-wells. After placing the seeded reconstituted ECM film into cell culture media, the HepG2 cells easily attached to the surface of the film. No obvious cytotoxicity was caused by the crosslinked reconstituted ECM. The patterned micro-wells of the film were stable during the cell culture. After 24 h of culture, the HepG2 cells were fixed with 10% buffered formalin for 5 min, and phase contrast images of the cells were taken under a microscope.

### Example 6

### Cell Growth on a Porous ECM Scaffold

A porous reconstituted ECM scaffold was prepared as described above. The scaffolds were prepared using an ECM concentration of 0.5%, 1%, and 2% (w/v). Samples of the scaffold were cut into a disc shape 6 mm in diameter and 2 mm in height. All samples were sterilized with UV light and 70% ethanol. The scaffolds were extensively washed with PBS and then immersed in DMEM. HepG2 cells were seeded in the scaffolds at a density of 2×10⁵ cells per scaffold. The seeded scaffold discs were cultured at 37° C. for 3, 5, and 7 days.

After 7 days of culture, the HepG2 cells in the scaffold discs were observed by confocal microscopy after staining the samples with 2 μM calcein AM in PBS at 37° C. for 90 min Cells were observed attached to all surfaces of the scaffold discs, including in the pores within the interior of the discs.

The number of cells in the scaffold discs was analyzed using the MTT assay.

At 3, 5, and 7 days after seeding HepG2 cells, 0.5 mg/ml MTT in DMEM was added to each sample and incubated at 37° C. for 4 h. The MTT solution was removed and dimethyl sulfoxide was added to dissolve the converted dye. The absorbance at 560 nm was quantified with a plate reader. The results are shown in Table 1 below.

TABLE 1
Growth of HepG2 cells on a porous ECM scaffold

| Days in culture | 0.5% ECM | 1% ECM | 2% ECM |
|---|---|---|---|
| 3 | 1.8 × 10^{4 a} | 6 × 10⁴ | 5 × 10⁴ |
| 5 | 3.7 × 10⁴ | 1 × 10⁵ | 6 × 10⁴ |
| 7 | 5 × 10⁴ | 1.25 × 10⁵ | 8 × 10⁴ |

^{a} values are number of cells on porous ECM scaffold quantified by the MTT assay described above (n = 3)

The number of HepG2 cells increased with increasing time in culture on the reconstituted ECM scaffolds at all ECM concentrations tested.

### Example 7

### Cell Type-Specific Function of Hepatic Cells Grown on a Porous ECM Scaffold

The liver-specific function of HepG2 cells was assessed by measuring the rate of urea synthesis by the cells.

Urea synthesis was quantified by a diacetyl monoxime method known in the art. Briefly, samples of growth media from HepG2 cell cultures were collected daily and stored at 4° C. A 40 μL volume of each sample was added to 5 mL of urea nitrogen reagent (0.825 M sulfuric acid, 1.08 M phosphoric acid, 0.549 mM thiosemicarbazide, and 6.31 mM cadmium sulfate hydrate). 500 !IL of 2% 2,3-butanedione monoxime solution was mixed into the sample by inversion and the reaction was incubated at 100° C. The absorbance of each sample was measured at 540 nm.

A porous ECM scaffold was prepared as described in EXAMPLE 2 above having an ECM concentration of 1% (w/v).

A PLGA control scaffold was prepared by salt leaching. In brief, 2 g of PLGA (75:25 lactide:glycolide) was dissolved in 2 mL of dichloromethane. Two grams of sodium chloride was then added to the PLGA solution and the mixture left to dry for 2 days. The scaffold thus produced was immersed in deionized water for 2 days to leach out the sodium chloride. The salt-free scaffold was air-dried for 24 h.

HepG2 cells were seeded at 2×10⁵ cells on the porous ECM scaffold, the PLGA scaffold, and a standard tissue culture plate. Urea synthesis was quantified over a 7 day period. The results are shown in Table 2 below.

TABLE 2
Secretion of urea by HepG2 cells

| | Porous ECM | PLGA | |
|---|---|---|---|
| Days in culture | scaffold | scaffold | Tissue culture plate |
| 3 | 750^{a} | 670 | 200 |
| 5 | 1000 | 1000 | 190***^{, ###} |
| 7 | 750 | 500* | 190***^{, #} |

^{a}values are expressed as μg urea/day/10⁶ cells (n = 4).
*= p > 0.05,
***p > 0.001, significantly different from porous ECM scaffold.
^{#} = p > 0.05,
^{###} = p > 0.001, significantly different from PLGA.

The amount of urea produced by HepG2 cells on day 5 and day 7 standardized by the number of cells was significantly greater than that produced by cells grown on a tissue culture plate. Unexpectedly, the amount of urea produced by HepG2 cells on day 7 was significantly greater than that produced by cells grown on a PLGA scaffold.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, a person skilled in the art can easily ascertain the essential characteristics of the present invention, and without departing from the spirit and scope thereof, can make various changes and modifications of the present invention to adapt it to various usages and conditions. Thus, other embodiments are also within the claims.

1. A microfabricated multi-tissue system for in vitro drug toxicity testing, comprising a plurality of layers, each of which is formed of decellularized tissue extracellular matrix (ECM) and has a thickness of 10 μm to 2 mm, wherein at least a first layer has parenchymal cells attached thereto and at least a second layer has non-parenchymal cells attached thereto, each layer contains only a single cell type, and the first layer is stacked on the second layer such that the non-parenchymal cells extend survival of the parenchymal cells and maintain a differentiated state thereof.

2. The microfabricated multi-tissue system of claim 1, wherein the parenchymal cells are hepatocytes, cardiomyocytes, kidney epithelial cells, enterocytes, beta cells, or cortical neurons.

3. The microfabricated multi-tissue system of claim 1, wherein the non-parenchymal cells are macrophages, fibroblasts, epithelial cells, adipocytes, or endothelial cells.

4. The microfabricated multi-tissue system of claim 1, wherein the plurality of layers are arranged in a tubular structure having a lumen and an exterior surface.

5. The microfabricated multi-tissue system of claim 4, wherein the ECM is decellularized tissue from lung, liver, heart, kidney, intestine, tendon, pancreas, brain, skin, fat, cartilage, spleen, bone, or tumor.

6. The microfabricated multi-tissue system of claim 5, wherein the parenchymal cells are hepatocytes and the non-parenchymal cells are fibroblasts.

7. An in vitro method for drug toxicity testing, the method comprising obtaining the microfabricated multi-tissue system of claim 1, exposing the system to a test drug, and determining a viability of the parenchymal cells or an expression level of tissue-specific genes in the parenchymal cells after exposure to the test drug, wherein the test drug is toxic if the viability of the parenchymal cells is reduced after exposure to the test drug or the expression level of tissue-specific genes is reduced as compared to a predetermined control expression level.

8. The method of claim 7, wherein the non-parenchymal cells are fibroblasts.

9. The method of claim 7, wherein the plurality of layers are arranged in a tubular structure having a lumen and an exterior surface and the system is exposed to the test drug via the lumen.

10. A method for producing a three dimensional co-culture system, the method comprising slicing a tissue sample into a plurality of sections each having a thickness of 10 μm to 2 mm, decellularizing the plurality of sections to form a plurality of sheets, seeding at least one first sheet with parenchymal cells, seeding at least one second s sheet with non-parenchymal cells, culturing the seeded sheets, stacking the cultured seeded sheets to form a flat tube precursor, and rolling the flat tube precursor to form a tubular structure having a lumen and an exterior surface, wherein each sheet is seeded with a single cell type.

11. The method of claim 10, wherein the parenchymal cells are hepatocytes and the non-parenchymal cells are macrophages, fibroblasts, epithelial cells, adipocytes, or endothelial cells.

12. The method of claim 10, wherein the tissue is lung, liver, heart, kidney, intestine, tendon, pancreas, brain, skin, fat, cartilage, spleen, bone, or tumor.

13. A method for producing a reconstituted extracellular matrix (ECM) construct, the method comprising obtaining a decellularized ECM from a tissue, homogenizing the decellularized ECM to form a paste, and casting the paste into a mold, thereby forming a reconstituted ECM construct.

14. The method of claim 13, further comprising mixing the paste with a cross-linking agent to form a mixture and, after the casting step, incubating the paste in the mold to effect cross-linking.

15. The method of claim 14, wherein the cross-linking agent is transglutaminase, glutaraldehyde, genipin, or 1-(3-dimethylaminopropyl)-3-ethylcarbodiimide.

16. The method of claim 14, further comprising, after the homogenizing step, incubating the paste with a proteolytic enzyme.

17. The method of claim 13, wherein the tissue is lung, liver, heart, kidney, intestine, tendon, pancreas, brain, skin, fat, cartilage, spleen, bone, or tumor.

18. The method of claim 17, wherein the mold is a flat paraffin mold or a polydimethylsiloxane micropatterned mold.

19. A reconstituted ECM film produced by the method of claim 13.

20. A reconstituted ECM film produced by the method of claim 14.

21. A reconstituted ECM film produced by the method of claim 17.

22-31. (canceled)
